(19) United States
(12) Patent Application Publication
(10) Pub. No.: US 2006/0019338 A1
(43) Pub. Date: Jan. 26, 2006

(54) Primate prokineticin and prokineticin receptor polypeptides, related compositions and methods

(76) Inventor: Qun-Yong Zhou, Irvine, CA (US)

Correspondence Address: LISA A. HAILE, J.D., Ph.D;DLA PIPER RUDNICK GRAY CARY US LLP, 4365 Executive Drive, Suite 1100, San Diego, CA, 92121-2133, US

(21) Appl. No.: 11/073,420

(22) Filed: Mar. 4, 2005

Related U.S. Application Data
(60) Provisional application No. 60/550,753, filed on Mar. 5, 2004.

(51) Int. Cl. C07K 14/705; C07H 21/04; C12P 21/06; C12N 5/06

(52) U.S. Cl. 435/69.1; 435/320.1; 435/363; 530/350; 536/23.5

(57) ABSTRACT

The invention provides an isolated squirrel monkey prokineticin receptor 2 (PKR2) polypeptide containing the amino acid sequence referenced as SEQ ID NO:2; an isolated chimpanzee PKR2 containing the amino acid sequence referenced as SEQ ID NO:4; and an isolated rhesus monkey prokineticin receptor 1 (PKR1) referenced as SEQ ID NO:30. Also provided are methods of identifying PKR1 and PKR2 agonists and antagonists using the newly identified PKR2 and PKR1 polypeptides. Additionally, the invention provides an isolated rhesus monkey PK2 polypeptide containing the amino acid sequence referenced as SEQ ID NO:6, and an isolated rhesus monkey PK1 polypeptide containing the amino acid sequence referenced as SEQ ID NO:28. Nucleic acid molecules encoding the disclosed polypeptides further are provided by the invention.

### CLAIM FOR PRIORITY

This application claims benefit of priority from U.S. Provisional Application Ser. No. 60/550,753, filed Mar. 5, 2004, the entire contents of which are incorporated herein by reference.

### BACKGROUND OF THE INVENTION

Prokineticins are secreted proteins that have roles in several biological functions, including circadian rhythm; angiogenesis; gastric contractility and motility; gastric acid and pepsinogen secretion; pain; and neurogenesis. Prokineticin 1 (PK1) and prokineticin 2 (PK2) induce cellular responses by binding to G-protein coupled receptors termed prokineticin receptor 1 (PKR1) and prokineticin receptor 2 (PKR2), resulting in activation of receptor signaling. Normal prokineticin receptor signaling contributes to the development and function of a variety of tissues in humans. If this normal signaling is disrupted, for example, due to disease, unwanted changes can occur at the cellular, tissue and whole organism level. These changes can be manifested in a variety of conditions and diseases associated with improper prokineticin receptor signaling.

To treat conditions associated with improper prokineticin receptor signaling, it is desirable to identify drugs that alter receptor activity to obtain a normal or otherwise optimal amount of signaling. Such drugs can be used, for example, to increase receptor signaling in individuals having conditions associated with insufficient prokineticin receptor activity or to decrease receptor signaling in individuals having conditions associated with excessive prokineticin receptor activity. Therefore, the identification of prokineticin receptor modulating drugs is expected to provide relief to individuals suffering from a variety of conditions attributed, at least in part, to insufficient or excessive prokineticin receptor signaling.

Thus, there exists a need to identify compounds and methods for modulating prokineticin receptor signaling. The invention satisfies this need and provides related advantages as well.

### SUMMARY OF THE INVENTION

The invention provides an isolated squirrel monkey prokineticin receptor 2 (PKR2) polypeptide containing the amino acid sequence referenced as

SEQ ID NO:2. Also provided by the invention is an isolated chimpanzee PKR2 containing the amino acid sequence referenced as SEQ ID NO:4. Further provided is a method of identifying a PKR2 agonist using the squirrel monkey PKR2 or chimpanzee PKR2. The method involves (a) contacting a PKR2 polypeptide containing an amino acid sequence referenced as SEQ ID NO:2 or SEQ ID NO:4 with one or more candidate compounds, and (b) identifying a compound that selectively promotes production of a PKR2 signal, said compound being characterized as an agonist of said PKR2.

The invention also provides a method of identifying a PKR2 antagonist. The method involves (a) contacting a PKR2 polypeptide containing an amino acid sequence referenced as SEQ ID NO:2 or SEQ ID NO:4, with one or more candidate compounds in the presence of a prokineticin, and (b) identifying a compound that selectively inhibits production of a PKR2 signal, said compound being characterized as an antagonist of said PKR2.

The invention provides an isolated nucleic acid molecule encoding a squirrel monkey PKR2 polypeptide, which contains the nucleotide sequence referenced as SEQ ID NO:1. Also provided is an isolated nucleic acid molecule encoding a chimpanzee PKR2 polypeptide, which contains the nucleotide sequence referenced as SEQ ID NO:3. Further provided are expression vectors containing a squirrel monkey or chimpanzee PKR2 nucleic acid molecule operatively linked to a promoter of gene expression. The invention also provides a host cell containing a squirrel monkey or chimpanzee PKR2 nucleic acid molecule-containing expression vector.

The invention provides an isolated rhesus monkey prokineticin 2 (PK2) polypeptide that contains the amino acid sequence referenced as SEQ ID NO:6. Also provided is an isolated nucleic acid molecule encoding the rhesus monkey PK2 polypeptide, which contains the nucleotide sequence referenced as SEQ ID NO:5. Additionally provided is an expression vector containing this nucleic acid molecule, operatively linked to a promoter of gene expression. A host cell containing the rhesus monkey PK2 expression vector is further provided.

The invention provides an isolated rhesus monkey prokineticin receptor 1 (PKR1) polypeptide containing the amino acid sequence referenced as

SEQ ID NO:30. Further provided is a method of identifying a PKR1 agonist using the rhesus monkey PKR1. The method involves (a) contacting a PKR1 polypeptide containing an amino acid sequence referenced as SEQ ID NO:30 with one or more candidate compounds, and (b) identifying a compound that selectively promotes production of a PKR1 signal, said compound being characterized as an agonist of said PKR1.

The invention also provides a method of identifying a PKR1 antagonist. The method involves (a) contacting a PKR1 polypeptide containing an amino acid sequence referenced as SEQ ID NO:30, with one or more candidate compounds in the presence of a prokineticin, and (b) identifying a compound that selectively inhibits production of a PKR1 signal, said compound being characterized as an antagonist of said PKR1.

The invention provides an isolated rhesus monkey prokineticin 1 (PK1) polypeptide that contains the amino acid sequence referenced as SEQ ID NO:28. Also provided is an isolated nucleic acid molecule encoding the rhesus monkey PK1 polypeptide, which contains the nucleotide sequence referenced as SEQ ID NO:27. Additionally provided is an expression vector containing this nucleic acid molecule, operatively linked to a promoter of gene expression. A host cell containing the rhesus monkey PK1 expression vector is further provided.

### BRIEF DESCRIPTION OF THE DRAWINGS

FIG. 1A shows the nucleotide sequence of squirrel monkey PKR2 (SEQ ID NO:1). FIG. 1B shows the amino acid sequence of squirrel monkey PKR2 (SEQ ID NO:2).

FIG. 2A shows the nucleotide sequence of chimpanzee PKR2 (SEQ ID NO:3). FIG. 2B shows the amino acid sequence of chimpanzee PKR2 (SEQ ID NO:4).

FIG. 3 shows a comparison of PKR2 amino acid sequences from squirrel monkey (SEQ ID NO:2), human

(SEQ ID NO:7), M. Fascicularis (SEQ ID NO:8) and chimpanzee (SEQ ID NO:4).

FIG. 4A shows the nucleotide sequence of rhesus monkey PK2 (SEQ ID NO:5). FIG. 4B shows the amino acid sequence of rhesus monkey PK2 (SEQ ID NO:6). The signal peptide portion of the PK2 amino acid sequence is underlined.

FIG. 5 shows a comparison of PK2 amino acid sequences from rhesus monkey (SEQ ID NO:6), human (SEQ ID NO:9), and mouse (SEQ ID NO:10).

FIG. 6 shows amino acid sequences of human PK1 (SEQ ID NO:11); mouse PK1 (SEQ ID NO:12); toad BV8 (SEQ ID NO:13); frog BV8 (SEQ ID NO:14); snake MIT1 (SEQ ID NO:15); human PK1-PK2 chimera (SEQ ID NO:16); and human PK2-PK1 chimera (SEQ ID NO:17).

FIG. 7A shows the nucleotide sequence of rhesus monkey PK1 (SEQ ID NO:27). FIG. 7B shows the amino acid sequence of rhesus monkey PK1 (SEQ ID NO:28). The signal peptide portion of the PK1 amino acid sequence is underlined.

FIG. 8 shows a comparison of PK1 amino acid sequences containing signal peptides from rhesus monkey (SEQ ID NO:29) and human (SEQ ID NO:31. The residues that differ between the rhesus monkey and human sequences are shown in bold.

FIG. 9A shows the nucleotide sequence of rhesus monkey PKR1 (SEQ ID NO:29). FIG. 9B shows the amino acid sequence of rhesus monkey PKR1 (SEQ ID NO:30).

FIG. 10 shows a comparison of PKR1 amino acid sequences from rhesus monkey (SEQ ID NO:30 and human (SEQ ID NO:32). The residues of rhesus monkey PKR1 that differ from the human PKR1 sequence are shown in bold.

FIG. 11 shows activation of squirrel monkey PKR2 and human PKR2 in response to PK1, as demonstrated by an increase in calcium mobilization.

FIG. 12 shows activation of rhesus monkey PKR1 and human PKR1 in response to PK2, as demonstrated by an increase in calcium mobilization

### DETAILED DESCRIPTION OF THE INVENTION

The invention provides newly identified primate prokineticin receptor 1 (PKR1) and prokineticin receptor 2 (PKR2) polypeptides, prokineticin 1 (PK1) and prokineticin 2 (PK2) polypeptides, as well as encoding nucleic acid molecules. In particular, PKR2 polypeptides from squirrel monkey and chimpanzee; PKR1 polypeptide from rhesus monkey; and PK1 and PK2 polypeptides from rhesus monkey are provided. The PKR1, PKR2, PK1 and PK2 polypeptides of the invention can be used, for example, in screening methods for identifying prokineticin receptor modulating compounds, including receptor agonists and antagonists.

Agonists and antagonists identified using the methods of the invention can be beneficially used modulate prokineticin (PK) receptor activity in an individual to treat a condition associated with aberrant low or high level of PKR1 or PKR2 activity. For example, because PK receptors can mediate circadian rhythm function in animals (Cheng et al. Nature 247:405-410 (2002)), a PK receptor modulating compound can be used to treat disorders of circadian rhythm function, such as sleep disorders, shift work disorders, seasonal depression and jet lag.

Further, because PK receptors can mediate angiogenesis in a variety of tissues (LeCouter et al., Nature 412:877-884 (2001); Lin et al. J. Biol. Chem. 277:19 (2002)), including endothelium, a PK receptor antagonist can be used to reduce or inhibit angiogenesis in prokineticin receptor expressing tissues. Such an antagonist can be useful for treating cancer and female reproductive disorders such as menorrhagia, endometriosis, dysfunctional uterine bleeding, fibroids and adenoyosis.

Also, because PK receptors can mediate gastric contractility and motility, as well as mediate secretion of gastric acid and pepsinogen, a PK receptor modulating compound can be used to treat, for example, gastric reflux disorder (GERD) irritable bowel syndrom, postoperative ileus, diabetic gastroparesis, chronic constipation and can be used for reducing gastrointestinal side effects of chemotherapy. Moreover, because PK receptors can mediate neurogenesis, a PK receptor receptor modulating compound can be used to treat neurological disorders, including those induced by stroke and trauma. Other indications that can be treated using a PK receptor modulating compound include ischemic heart disease, critical limb ischemia, wound healing and burns, cancer, diabetic retinopathy, and inflammatory diseases such as arthritis and psoriasis.

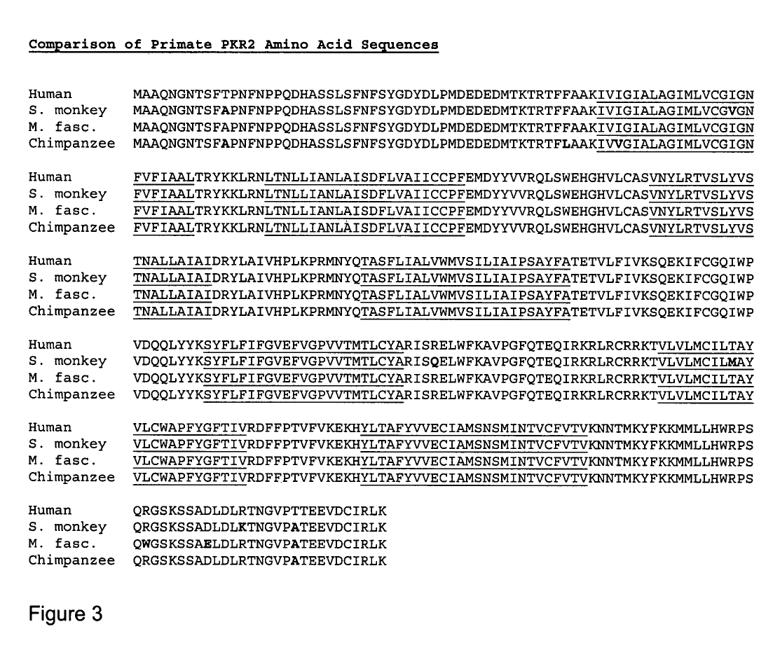

The squirrel monkey PKR2 amino acid sequence (SEQ ID NO:2) disclosed herein differs from known primate PKR2 amino acid sequences at several amino acid positions, as is shown in FIG. 3. For example, in comparison to human PKR2 (GenBank AF506288), the squirrel monkey PKR2 sequence contains an alanine residue rather than a threonine at position 11, a valine residue rather than an isoleucine at position 69, a glutamine residue rather than an arginine at position 248, a methionine residue rather than threonine at position 282, a lysine residue rather than an arginine at position 368, and an alanine residue rather than a threonine at position 374; in comparison to Cercopithecus aethiops (vervet monkey) PKR2 (U.S. Patent Application Publication 0030059856), which appears to be identical to Macaca Fascicularis (long-tailed macaque) PKR2 (PCT publication WO 01/53308), the squirrel monkey PKR2 amino acid sequence (SEQ ID NO:2) contains a valine residue rather than an isoleucine at position 69, a glutamine residue rather than an arginine at position 248, a methionine residue rather than threonine at position 282, an arginine residue rather than a tryptophan at position 357, an aspartic acid residue rather than a glutamic acid at position 364, and a lysine residue rather than an arginine at position 368. Thus, the disclosed squirrel monkey PKR2 amino acid sequence differs from both human and vervet monkey/macaque PKR2 amino acid sequences at multiple amino acid positions.

The chimpanzee PKR2 amino acid sequence (SEQ ID NO:4) disclosed herein differs from known primate PKR2 amino acid sequences in several amino acid positions, also as is shown in FIG. 3. For example, in comparison to human PKR2 (GenBank AF506288), the chimpanzee PKR2 sequence contains an alanine residue rather than a threonine at position 11, a leucine residue rather than a phenylalanine at position 50, a valine residue rather than an isoleucine at position 56, and an alanine residue rather than a threonine at position 374; in comparison to Cercopithecus aethiops (vervet monkey) PKR2 (U.S. Patent Application Publication 0030059856), which appears to be identical to Macaca Fascicularis (long-tailed macaque) PKR2 (PCT publication WO 01/53308), the disclosed chimpanzee PKR2 sequence contains a leucine residue rather than a phenylalanine at position 50, a valine residue rather than an isoleucine at position 56, an arginine residue rather than a tryptophan at position 357, and an aspartic acid residue rather than a glutamic acid at position 364. Thus, the disclosed chimpanzee PKR2 amino acid sequence differs from both human and vervet monkey/macaque PKR2 amino acid sequences at multiple amino acid positions.

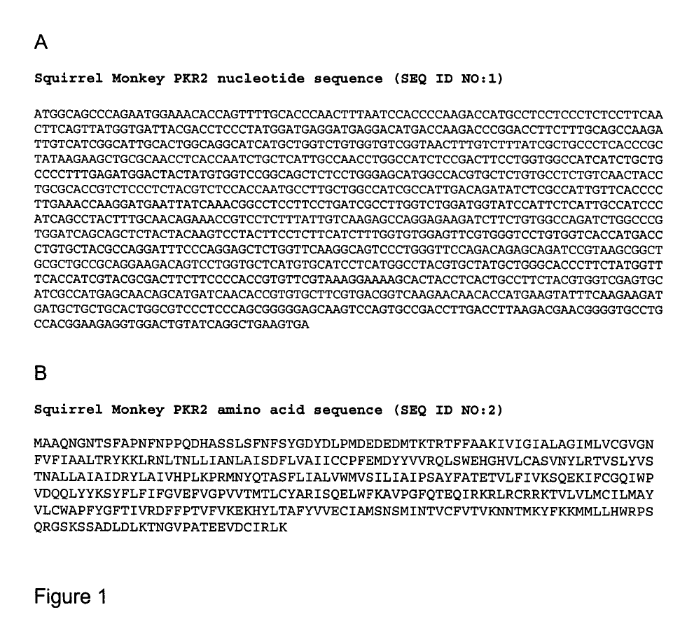

In an embodiment, the invention provides an isolated squirrel monkey PKR2 polypeptide containing the amino acid sequence referenced as SEQ ID NO:2. As used herein, the term “prokineticin receptor 2” or “PKR2” refers to a heptahelical membrane-spanning polypeptide that binds to a prokineticin and signals through a G-protein coupled signal transduction pathway in response to prokineticin binding. The terms “squirrel monkey prokineticin receptor 2” and “squirrel monkey PKR2” refer to a polypeptide comprising the amino acid sequence of squirrel monkey PKR2 shown in FIG. 1B (SEQ ID NO:2).

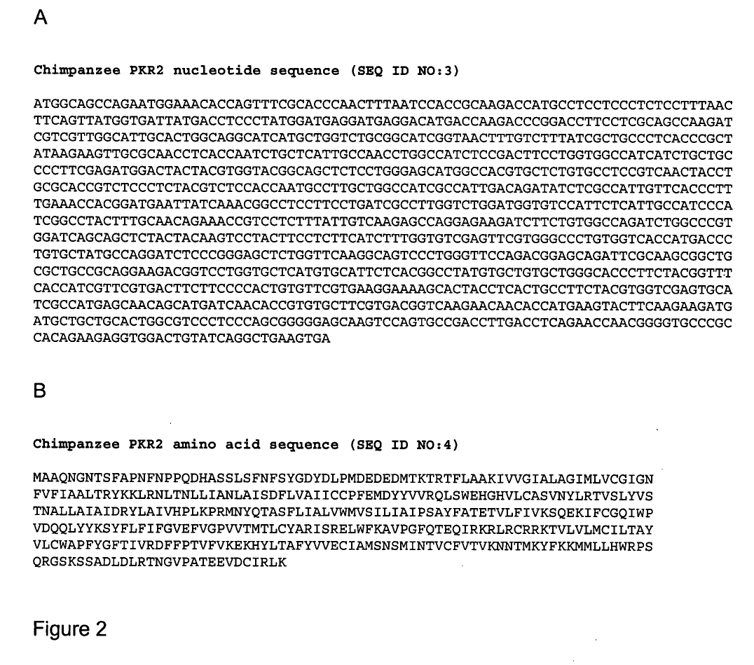

In another embodiment, the invention provides an isolated chimpanzee PKR2 polypeptide containing the amino acid sequence referenced as SEQ ID NO:4. The terms “chimpanzee prokineticin receptor 2,” “chimpanzee PKR2, “Pan troglodytes PKR2,” and “P. troglodytes PKR2” refer to a polypeptide comprising the amino acid sequence of chimpanzee PKR2 shown in FIG. 2B (SEQ ID NO:4).

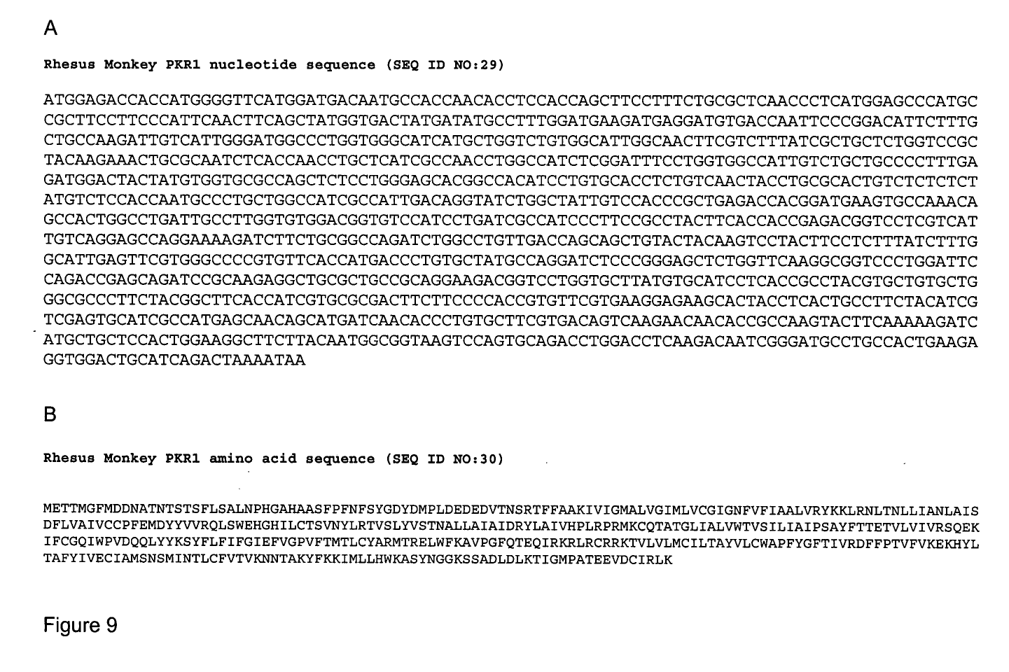

In an embodiment, the invention provides an isolated rhesus monkey PKR1 polypeptide containing the amino acid sequence referenced as SEQ ID NO:30. As used herein, the term “prokineticin receptor 1” or “PKR1” refers to a heptahelical membrane-spanning polypeptide that binds to a prokineticin and signals through a G-protein coupled signal transduction pathway in response to prokineticin binding. The terms “rhesus monkey prokineticin receptor 1” and “rhesus monkey PKR1” refer to a polypeptide comprising the amino acid sequence of rhesus monkey PKR1 shown in FIG. 9B (SEQ ID NO:30).

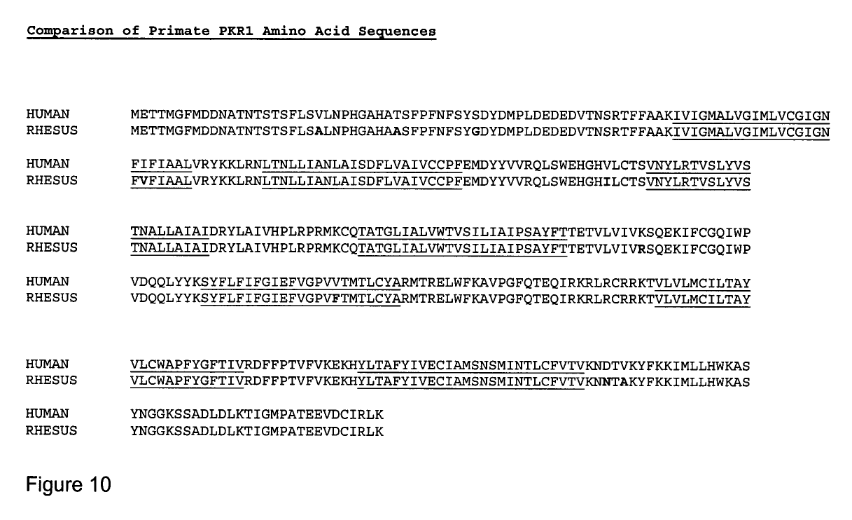

The rhesus monkey PKR1 amino acid sequence (SEQ ID NO:30) disclosed herein differs from the human PKR1 amino acid sequence at several amino acid positions, as is shown in FIG. 10. For example, in comparison to human PKR1 (GenBank AF506287; FIG. 10), the rhesus monkey PKR1 sequence contains an alanine residue rather than a valine at position 22, an alanine residue rather than a threonine at position 31, a glycine residue rather than a serine at position 40, a valine residue rather than an isoleucine at position 82, an isoleucine residue rather than a valine at position 135, an arginine residue rather than a lysine at position 300, an asparagine residue rather than an aspartic acid at position 438, and an alanine residue rather than a valine at position 440. Thus, the disclosed rhesus monkey PKR1 amino acid sequence differs from the human PKR1 amino acid sequences at nine amino acid positions.

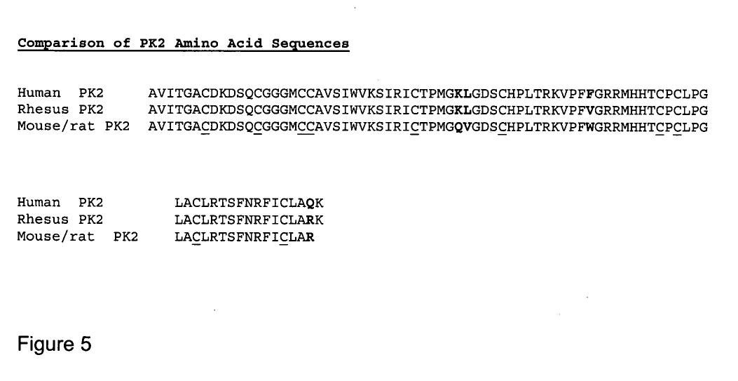

In a further embodiment, the invention provides an isolated rhesus monkey PK2 polypeptide containing the amino acid sequence referenced as SEQ ID NO:6. The rhesus monkey PK2 amino acid sequence disclosed herein differs from known PK2 amino acid sequences at several amino acid positions, as is shown in FIG. 5. For example, in comparison to human PK2 (see GenBank NM 021935 and Sheppard et al. U.S. Pat. No. 6,485,938), the rhesus monkey PK2 sequence contains a valine residue rather than a phenylalanine at position 51, and an arginine residue rather than a glutamine at position 79; in comparison to mouse/rat PK2 (GenBank AF 487280), the disclosed rhesus monkey PK2 sequence contains a lysine residue rather than a glutamine at position 36, a leucine residue rather than a valine at position 37, and a valine residue rather than a tryptophan at position 51. Thus, the disclosed rhesus monkey PK2 amino acid sequence differs from both human and mouse/rat PK2 at multiple amino acid positions.

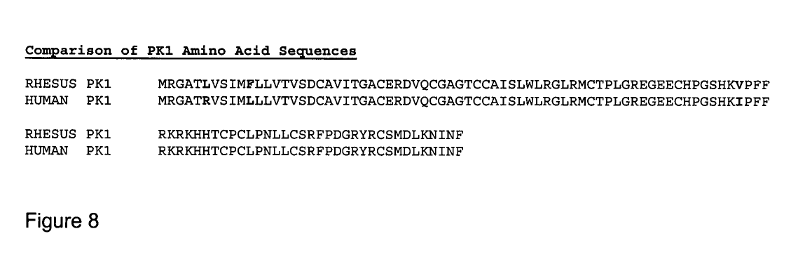

In a further embodiment, the invention provides an isolated rhesus monkey PK1 polypeptide containing the amino acid sequence referenced as SEQ ID NO:28. The rhesus monkey PK1 amino acid sequence disclosed herein differs from human PK1 at several amino acid positions, as is shown in FIG. 8. For example, in comparison to human PK1 containing the signal peptide (SEQ ID NO:31; FIG. 8), the rhesus monkey PK1 sequence contains a leucine residue rather than an arginine at position 6, a phenylalanine residue rather than a leucine at position 11, and a valine residue rather than an isoleucine at position 67. Thus, the disclosed rhesus monkey PK1 amino acid sequence differs from human PK1 at three amino acid positions.

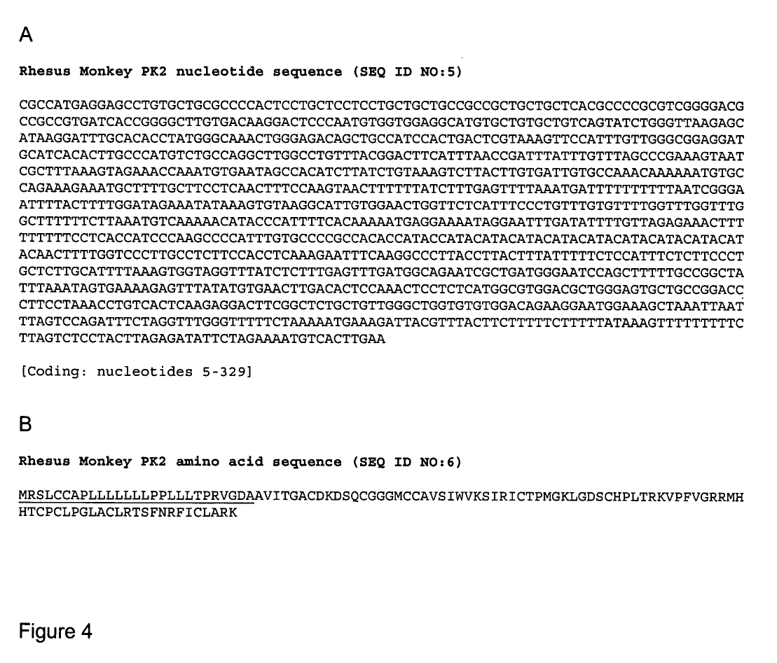

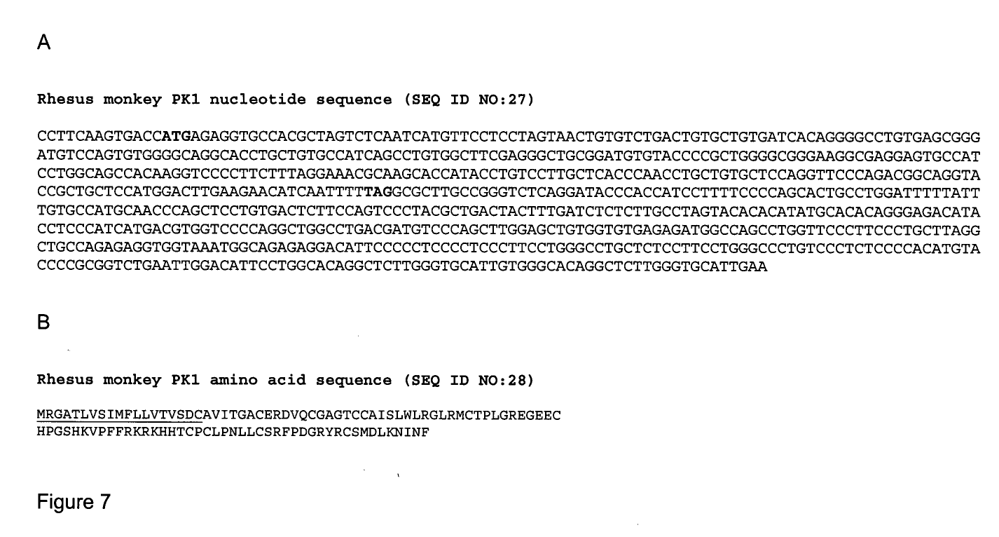

As used herein, the term “prokineticin” or “PK” refers to a peptide that binds to a prokineticin receptor and elicits signaling by the receptor through a G-protein coupled signal transduction pathway. The term “rhesus monkey PK2 polypeptide” refers to a polypeptide comprising the amino acid sequence of rhesus prokineticin 2 shown as the non-underlined sequence in FIG. 4B (SEQ ID NO:6) and to a fragment of the reference polypeptide that has prokineticin activity. The term “rhesus monkey PK1 polypeptide” refers to a polypeptide comprising the amino acid sequence of rhesus prokineticin 1 shown as the non-underlined sequence in FIG. 7B (SEQ ID NO:28) and to a fragment of the reference polypeptide that has prokineticin activity.

Because of the homology between PKR2 and PKR1, which have amino acid sequences that are about 85% identical, a PK2 or PK1 polypeptide of the invention can function as a PKR2 or PKR1 agonist. Thus, a PK2 or PK1 polypeptide of the invention can be used as a PKR2 or PKR1 agonist in the screening methods of the invention as well as in a variety of applications in which PKR1 or PKR2 activation is desired.

A polypeptide of the invention can contain a reference amino acid sequence, such as SEQ ID NO:2, 4, 6, 28 or 30 together with a heterologous amino acid sequence. Examples of heterologous amino acid sequences include purification tags, detection tags, and cell localization tags. Non-limiting examples of polypeptide tags that can be contained in a polypeptide of the invention include GST tags, His tags, Flag tags, Myc tags, hemagglutinin tags, multiple affinity purification (MAFT), and tandem affinity purification (TAP) tags.

The invention provides an isolated nucleic acid molecule comprising a sequence that encodes squirrel monkey PKR2 amino acid sequence SEQ ID NO:2. The squirrel monkey PKR2 nucleic acid molecule of the invention can have nucleotide sequence SEQ ID NO:1, or substantially the same nucleotide sequence as SEQ ID NO:1, or a fragment thereof, and optionally can contain a heterologous sequence, such as a tag. Further provided is an isolated nucleic acid molecule comprising a sequence that encodes chimpanzee PKR2 amino acid sequence SEQ ID NO:4. The chimpanzee PKR2 nucleic acid molecule of the invention can have nucleotide sequence SEQ ID NO:3, or substantially the same nucleotide sequence as SEQ ID NO:3, or a fragment thereof, and optionally can contain a heterologous sequence, such as a tag.

The invention provides an isolated nucleic acid molecule comprising a sequence that encodes rhesus monkey PKR1 amino acid sequence SEQ ID NO:30. The rhesus monkey PKR1 nucleic acid molecule of the invention can have nucleotide sequence SEQ ID NO:29, or substantially the same nucleotide sequence as SEQ ID NO:29, or a fragment thereof, and optionally can contain a heterologous sequence, such as a tag.

The invention also provides an isolated nucleic acid molecule comprising a sequence that encodes rhesus monkey PK2 amino acid sequence SEQ ID NO:6. The rhesus monkey PK2 nucleic acid molecule of the invention can have nucleotide sequence SEQ ID NO:5, or substantially the same nucleotide sequence as SEQ ID NO:5, or a fragment thereof, and optionally can contain a heterologous sequence, such as a tag.

Further provided by the invention is an isolated nucleic acid molecule comprising a sequence that encodes rhesus monkey PK1 amino acid sequence SEQ ID NO:28. The rhesus monkey PK1 nucleic acid molecule of the invention can have nucleotide sequence SEQ ID NO:27, or substantially the same nucleotide sequence as SEQ ID NO:27, or a fragment thereof, and optionally can contain a heterologous sequence, such as a tag.

A nucleic acid molecule of the invention can be linked to a variety of heterologous nucleotide sequences, which can encode a heterologous polypeptide or peptide if desired. Exemplary heterologous nucleotide sequences include, a restriction site, a promoter or other regulatory element, a detection tag, an a nucleotide sequence that encodes a tag or other useful sequence in the polypeptide. Non-limiting examples of such tags include a purification tag useful in the isolation of the encoded polypeptide and a detection tag useful in the detection of the encoded polypeptide.

As used herein, the term “nucleic acid molecule” refers to a polynucleotide, including an oligonucleotide, of natural or synthetic origin, which can be single- or double-stranded, can correspond to genomic DNA, cDNA or RNA, and can represent either the sense or antisense strand or both.

The term “nucleic acid molecule” is intended to include nucleic acid molecules that contain one or more non-natural nucleotides, such as nucleotides having modifications to the base, the sugar, or the phosphate portion, or having one or more non-natural linkages, such as phosphorothioate linkages. Such modifications can be advantageous in increasing the stability of the nucleic acid molecule, particularly when used in hybridization applications.

Furthermore, the term “nucleic acid molecule” is intended to include nucleic acid molecules modified to contain a detectable moiety, such as a radiolabel, a fluorochrome, a ferromagnetic substance, a luminescent tag or a detectable binding agent such as biotin. Nucleic acid molecules containing such moieties are useful as probes for detecting the presence or expression of a PKR2 nucleic acid molecule.

As used herein, the term “isolated nucleic acid molecule” is intended to mean that the nucleic acid molecule is altered, by the hand of man, from how it is found in its natural environment. For example, an isolated nucleic acid molecule can be a molecule operatively linked to an exogenous nucleic acid sequence. An isolated nucleic acid molecule can also be a molecule removed from some or all of its normal flanking nucleic acid sequences.

The invention provides vectors that contain a nucleic acid molecule of the invention, and isolated host cells containing the vector. Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. The vectors of the invention will generally contain elements such as an origin of replication compatible with the intended host cells; transcription termination and RNA processing signals; one or more selectable markers compatible with the intended host cells; and one or more multiple cloning sites. Optionally, the vector can further contain heterologous sequences encoding tag sequences, such as GST tags, and/or a protease cleavage site, such as a Factor Xa site, which facilitate expression and purification of the encoded polypeptide.

The choice of particular elements to include in a vector will depend on factors such as the intended host cells; the insert size; whether expression of the inserted sequence is desired; the desired copy number of the vector; the desired selection system, and the like. The factors involved in ensuring compatibility between a host cell and a vector for different applications are well known in the art.

In applications in which the vectors are to be used for recombinant expression of the encoded polypeptide, the isolated nucleic acid molecules will generally be operatively linked to a promoter of gene expression, which may be present in the vector or in the inserted nucleic acid molecule. An isolated nucleic acid molecule encoding a squirrel monkey PKR2 of the invention, a chimpanzee PKR2 of the invention, a rhesus monkey PKR1, rhesus monkey PK1 or rhesus monkey PK2 of the invention can be operatively linked to a promoter of gene expression. As used herein, the term “operatively linked” means that the nucleic acid molecule is positioned with respect to either the endogenous promoter, or a heterologous promoter, in such a manner that the promoter will direct the transcription of RNA using the nucleic acid molecule as a template.

Methods for operatively linking a nucleic acid to a heterologous promoter are well known in the art and include, for example, cloning the nucleic acid into a vector containing the desired promoter, or appending the promoter to a nucleic acid sequence using PCR. A nucleic acid molecule operatively linked to a promoter of RNA transcription can be used to express prokineticin transcripts and polypeptides in a desired host cell or in vitro transcription-translation system.

The choice of promoter to operatively link to an invention nucleic acid molecule will depend on the intended application, and can be determined by those skilled in the art. For example, if a particular gene product may be detrimental to a particular host cell, it may be desirable to link the invention nucleic acid molecule to a regulated promoter, such that gene expression can be turned on or off. Alternatively, it may be desirable to have expression driven by either a weak or strong constitutive promoter. Exemplary promoters suitable for mammalian cell systems include, for example, the SV40 early promoter, the cytomegalovirus (CMV) promoter, the mouse mammary tumor virus (MMTV) steroid-inducible promoter, and the Moloney murine leukemia virus (MMLV) promoter. Exemplary promoters suitable for bacterial cell systems include, for example, T7, T3, SP6, lac and trp promoters. An exemplary vector suitable for fusion protein expression in bacterial cells is the pGEX-3X vector (Amersham Pharmacia Biotech, Piscataway, N.J.).

The invention provides cells containing an isolated nucleic acid molecule encoding a polypeptide of the invention, such as a squirrel monkey PKR2 polypeptide containing SEQ ID NO:2 or fragment thereof, a chimpanzee PKR2 polypeptide containing SEQ ID NO:4 or fragment thereof, a rhesus monkey PKR1 polypeptide containing SEQ ID NO:30 or fragment thereof, a rhesus monkey PK1 polypeptide containing SEQ ID NO:28 or a fragment thereof, or a rhesus monkey PK2 polypeptide containing SEQ ID NO:6 or fragment thereof. The isolated nucleic acid molecule contained in the cells will generally be present within a vector, and can be maintained episomally, or incorporated into the host cell genome.

The cells of the invention can be used, for example, for molecular biology applications such as expansion, subcloning or modification of the isolated nucleic acid molecule. For such applications, bacterial cells, such as laboratory strains of E. coli, are useful, and expression of the encoded polypeptide is not required.

The cells of the invention can also advantageously be used to recombinantly express and isolate the encoded polypeptide. For such applications bacterial cells (for example, E. coli), insect cells (for example, Drosophila and Spodoptera fugiperda), yeast cells (for example, S. cerevisiae, S. pombe, or Pichia pastoris), and vertebrate cells (for example, mammalian primary cells and established cell lines, such as CHO, 293 and COS cells; and amphibian cells, such as Xenopus embryos and oocytes).

The invention also provides methods for preparing an isolated polypeptide corresponding to a squirrel monkey PKR2 polypeptide, chimpanzee PKR2 polypeptide, a rhesus monkey PKR1 polypeptide, rhesus monkey PK1 polypeptide, and a rhesus monkey PK2 polypeptide, by culturing host cells so as to express a recombinant polypeptide. A variety of well-known methods can be used to introduce a vector into a host cell for expression of a recombinant polypeptide (see, for example, Sambrook et al., Molecular Cloning: A Laboratory Manual, Cold Spring Harbor Laboratory, New York (1992) and Ansubel et al., Current Protocols in Molecular Biology, John Wiley and Sons, Baltimore, Md. (1998)). The selected method will depend, for example, on the selected host cells.

An isolated polypeptide of the invention can be prepared by biochemical procedures, and can be isolated from host cells that recombinantly express the polypeptide, or from tissues or cells that normally express the polypeptides. A variety of well-known biochemical procedures routinely used in the art, including membrane fractionation, chromatography, electrophoresis and ligand affinity methods, and immunoaffinity methods with the prokineticin antibodies described herein, can be used. An isolated polypeptide of the invention can also be prepared by chemical synthesis procedures known in the art. Following chemical synthesis, an inactive prokineticin can be refolded by the methods described herein to restore activity.

If desired, such as to optimize their functional activity, selectivity, stability or bioavailability, chemically synthesized polypeptides can be modified to include D-stereoisomers, non-naturally occurring amino acids, and amino acid analogs and mimetics. Examples of modified amino acids and their uses are presented in Sawyer, Peptide Based Drug Design, ACS, Washington (1995) and Gross and Meienhofer, The Peptides: Analysis, Synthesis, Biology, Academic Press, Inc., New York (1983). For certain applications, it can also be useful to incorporate one or more detectably labeled amino acids into a chemically synthesized polypeptide or peptide, such as radiolabeled or fluorescently labeled amino acids.

Methods of recombinantly expressing prokineticins are also known in the art (see US 20020115610A1 and Masuda et al., supra (2001) for examples of bacterial expression and WO 01/36465 and WO 00/52022 for examples of eukaryotic expression). Such methods can involve initially expressing the PK as a fusion protein, such as a fusion with a glutathione-S-transferase tag, Fc tag, 6×His tag, myc epitope, or other tag sequences known in the art. Methods of substantially purifying recombinantly expressed prokineticins, and for removing optional tag sequences, are also known in the art. For example, US 20020115610A1 describes conditions for refolding and purifying recombinantly expressed prokineticins that minimize protein aggregation, and also describes methods of confirming correct disulfide bond formation.

The compositions of the invention can also include crude or partially purified lysates or extracts of cells containing PKR1, PKR2, PK1, PK2, or more than one of these polypeptides, and reconstituted signaling systems containing PKR1, PKR2, PK1, PK2, or more than one of these polypeptides. Artificial signaling systems include, for example, natural or artificial lipid bilayers, such as a liposome or micelle, which promote an active conformation of PKR2 or PRK1. The compositions can further contain cellular fractions or isolated components necessary for producing and detecting a desired predetermined signal.

A composition of the invention further can contain a PKR2 or PKR1 and either or both PK1 and PK2, or another PK receptor agonist, such as a PK2/PK1 chimera or PK1/PK2 chimera.

As used herein, the term “isolated” indicates that the polypeptide is altered by the hand of man from how it is found in its natural environment. An “isolated” polypeptide of the invention can be a “substantially purified” molecule, that is at least 60%, 70%, 80%, 90 or 95% free from cellular components with which it is naturally associated. An isolated polypeptide can be in any form, such as in a buffered solution, a suspension, a lyophilized powder, recombinantly expressed in a heterologous cell, bound to a receptor or attached to a solid support.

The squirrel monkey and chimpanzee PKR2 polypeptides of the invention can be used in a variety of screening assays for identifying an antagonist or agonist of a PKR2. In one embodiment, the invention provides a method of identifying a PKR2 agonist. The method involves (a) contacting a PK2R containing an amino acid sequence referenced as SEQ ID NO:2 or SEQ ID NO:4 with one or more candidate compounds, and (b) identifying a compound that selectively promotes production of a PKR2 signal, the compound being characterized as an agonist of said PKR2.

In another embodiment, the invention provides a method of identifying a PKR2 antagonist. The method involves (a) contacting a PKR2 polypeptide comprising an amino acid sequence referenced as SEQ ID NO:2 or SEQ ID NO:4, with one or more candidate compounds in the presence of a prokineticin, and (b) identifying a compound that selectively inhibits production of a PKR2 signal, said compound being characterized as an antagonist of said PKR2.

As used herein, the terms “prokineticin receptor 2 antagonist” and “PKR2 antagonist” mean a compound that selectively inhibits or decreases normal signal transduction through a prokineticin receptor 2 (PKR2), which can be, for example, a squirrel monkey or chimpanzee PKR2 polypeptide of the invention. A PKR2 antagonist can act by any antagonistic mechanism, such as by binding a PKR2 or PK, thereby inhibiting binding between PK and PKR2. A PKR2 antagonist can also inhibit binding between a specific or non-specific PKR2 agonist and PKR2. Such a specific or non-specific PKR2 agonist can be, for example, a drug that produces unwanted side effects by promoting signaling through the PKR2. A PKR2 antagonist can also act, for example, by inhibiting the binding activity of PK or signaling activity of PKR2. For example, a PKR2 antagonist can act by altering the state of phosphorylation or glycosylation of PKR2. A PKR2 antagonist can also be an inverse agonist, which decreases PKR2 signaling from a baseline amount of constitutive PKR2 signaling activity.

As used herein, the terms “prokineticin receptor 2 agonist” and “PKR2 agonist” mean a compound that selectively promotes or enhances normal signal transduction through a prokineticin receptor 2 (PKR2), which can be, for example, a squirrel monkey or chimpanzee PKR2 polypeptide of the invention. A PKR2 agonist can act by any agonistic mechanism, such as by binding a prokineticin receptor at the normal prokineticin (PK) binding site, thereby promoting PKR2 signaling. A PKR2 agonist can also act, for example, by potentiating the binding activity of PK or signaling activity of PKR2. An agonist of a PKR2 also can function as an agonist of a PKR1 because PK1 and PK2 both can bind to PKR1 and PKR2. As such, a PKR1 agonist can be tested for its ability to function as a PKR2 agonist using the screening methods described herein; and a PKR2 agonist can be tested for its ability to function as a PKR1 agonist using the screening methods described herein.

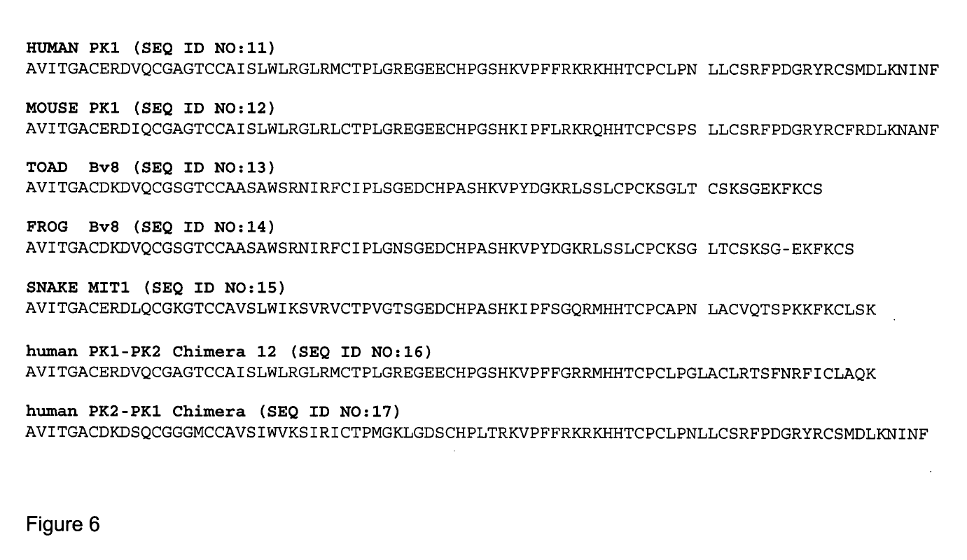

Specific examples of PKR2 agonists include the rhesus monkey, human, and mouse PK2 amino acid sequences shown in FIG. 5, the rhesus monkey PK1 amino acid sequence shown in FIG. 7B, as well as the human and mouse PK1 amino acid sequences (SEQ ID NOS:11 and 12, respectively), toad Bv8 amino acid sequence (SEQ ID NO:13), frog Bv8 amino acid sequence (SEQ ID NO:14), snake MIT1 amino acid sequence (SEQ ID NO:15), and chimeric PK1-PK2 amino acid sequences (SEQ ID NOS:16 and 17), as shown in FIG. 6.

The rhesus monkey PKR1 polypeptide of the invention can be used in a variety of screening assays for identifying an antagonist or agonist of a PKR1. In one embodiment, the invention provides a method of identifying a PKR1 agonist. The method involves (a) contacting a PKR1 containing the amino acid sequence referenced as SEQ ID NO:30 with one or more candidate compounds, and (b) identifying a compound that selectively promotes production of a PKR1 signal, the compound being characterized as an agonist of said PKR1.

In another embodiment, the invention provides a method of identifying a PKR1 antagonist. The method involves (a) contacting a PKR1 polypeptide comprising the amino acid sequence referenced as SEQ ID NO:30, with one or more candidate compounds in the presence of a prokineticin, and (b) identifying a compound that selectively inhibits production of a PKR1 signal, said compound being characterized as an antagonist of said PKR1.

As used herein, the terms “prokineticin receptor 1 antagonist” and “PKR1 antagonist” mean a compound that selectively inhibits or decreases normal signal transduction through a prokineticin receptor 1 (PKR1), which can be, for example, a rhesus monkey PKR1 polypeptide of the invention. A PKR1 antagonist can act by any antagonistic mechanism, such as by binding a PKR1 or PK, thereby inhibiting binding between PK and PKR1. A PKR1 antagonist can also inhibit binding between a specific or non-specific PKR1 agonist and PKR1. Such a specific or non-specific PKR1 agonist can be, for example, a drug that produces unwanted side effects by promoting signaling through the PKR1. A PKR1 antagonist can also act, for example, by inhibiting the binding activity of PK or signaling activity of PKR1. For example, a PKR1 antagonist can act by altering the state of phosphorylation or glycosylation of PKR1. A PKR1 antagonist can also be an inverse agonist, which decreases PKR1 signaling from a baseline amount of constitutive PKR1 signaling activity.

As used herein, the terms “prokineticin receptor 1 agonist” and “PKR1 agonist” mean a compound that selectively promotes or enhances normal signal transduction through a prokineticin receptor 1 (PKR1), which can be, for example, the rhesus monkey PKR1 polypeptide of the invention. A PKR1 agonist can act by any agonistic mechanism, such as by binding a prokineticin receptor at the normal prokineticin (PK) binding site, thereby promoting PKR1 signaling. A PKR1 agonist can also act, for example, by potentiating the binding activity of PK or signaling activity of PKR1.

An agonist or antagonist of a PKR1 also can function as an agonist or antagonist of a PKR2 because PK1 and PK2 both can bind to PKR1 and PKR2. As such, a PKR1 or PKR2 agonist or antagonist can be tested for its ability to function as a PKR2 or PKR1 agonist or antagonist using the screening methods described herein.

Specific examples of PKR1 agonists include the rhesus monkey PK1 amino acid sequence shown in FIG. 7B, rhesus monkey, human, and mouse PK2 amino acid sequences shown in FIG. 5, as well as the human and mouse PK1 amino acid sequences (SEQ ID NOS:11 and 12, respectively), toad Bv8 amino acid sequence (SEQ ID NO:13), frog Bv8 amino acid sequence (SEQ ID NO:14), snake MIT1 amino acid sequence (SEQ ID NO:15), and chimeric PK1-PK2 amino acid sequences (SEQ ID NOS:16 and 17), as shown in FIG. 6.

A screening assay used in a method of the invention for identifying a PKR1 or PKR2 agonist or antagonist can involve detecting a signal produced by a PKR1 or PKR2. As used herein, the term “receptor signal” is intended to mean a readout, detectable by any analytical means, that is a qualitative or quantitative indication of activation of G-protein-dependent signal transduction through PKR1 or PKR2. Assays used to determine such qualitative or quantitative activation of G-protein-dependent signal transduction through PKR1 or PKR2, are referred to below as “signaling assays.”

A signaling assay can be performed to determine whether a candidate compound is a PK receptor agonist or antagonist. In such an assay, a PK receptor, such as the squirrel monkey or chimpanzee PKR2 polypeptides or rhesus monkey PKR1 disclosed herein, is contacted with one or more candidate compounds under conditions wherein the PK receptor produces a signal in response to an agonist, such as PK1 or PK2. In response to PK receptor activation, a signal can increase or a decrease from an unstimulated PK receptor baseline signal. A signal can be an increasing signal, for example, when the amount of detected second messenger molecule is increased in response to PK receptor activation. A signal can be a decreasing signal, for example, when the detected second messenger molecule is destroyed, for example, by hydrolysis, in response to PK receptor activation.

Similarly, a signaling assay can be performed to determine whether a candidate compound is a PK receptor antagonist. In such a signaling assay, a PK receptor is contacted with one or more candidate compounds under conditions wherein the PK receptor produces a signal in response to an agonist, such as PK1 or PK2, and a compound is identified that reduces production of the signal.

Signaling through G proteins can lead to increased or decreased production or liberation of second messengers, including, for example, arachidonic acid, acetylcholine, diacylglycerol, cGMP, cAMP, inositol phosphate, such as inositol-1,4,5-trisphosphate, and ions, including Ca⁺⁺ ions; altered cell membrane potential; GTP hydrolysis; influx or efflux of amino acids; increased or decreased phosphorylation of intracellular proteins; or activation of transcription.

Various assays, including high throughput automated screening assays, to identify alterations in G-protein coupled signal transduction pathways are well known in the art. Various screening assay that measure Ca⁺⁺, cAMP, voltage changes and gene expression are reviewed, for example, in Gonzalez et al., Curr. Opin. in Biotech. 9:624-631 (1998); Jayawickreme et al., Curr. Opin. Biotech. 8:629-634 (1997); and Coward et al., Anal. Biochem. 270:2424-248 (1999). Yeast cell-based bioassays for high-throughput screening of drug targets for G-protein coupled receptors are described, for example, in Pausch, Trends in Biotech. 15:487-494 (1997). A variety of cell-based expression systems, including bacterial, yeast, baculovirus/insect systems and mammalian cells, useful for detecting G-protein coupled receptor agonists and antagonists are reviewed, for example, in Tate et al., Trends in Biotech. 14:426-430 (1996).

Assays to detect and measure G-protein-coupled signal transduction can involve first contacting a sample containing a PKR2 polypeptide of the invention, such as an isolated cell, membrane or artificial membrane, such as a liposome or micelle, with a detectable indicator. A detectable indicator can be any molecule that exhibits a detectable difference in a physical or chemical property in the presence of the substance being measured, such as a color change. Calcium indicators, pH indicators, and metal ion indicators, and assays for using these indicators to detect and measure selected signal transduction pathways are described, for example, in Haugland, Molecular Probes Handbook of Fluorescent Probes and Research Chemicals, Sets 20-23 and 25 (1992-94). For example, calcium indicators and their use are well known in the art, and include compounds like Fluo-3 AM, Fura-2, Indo-1, FURA RED, CALCIUM GREEN, CALCIUM ORANGE, CALCIUM CRIMSON, BTC, OREGON GREEN BAPTA, which are available from Molecular Probes, Inc., Eugene Oreg., and described, for example, in U.S. Pat. Nos. 5,453,517, 5,501,980 and 4,849,362. Exemplary methods for performing signaling assays for PK receptor activity are known in the art (see, for example, US 20020115610A1, which describes mobilization assays.)

If desired, a receptor signal other than Ca²⁺ influx can be used as the readout for PK receptor activation. The specificity of a G-protein for cell-surface receptors is determined by the C-terminal five amino acids of the Gα subunit. The nucleotide sequences and signal transduction pathways of different classes and subclasses of Gα subunits in a variety of eukaryotic and prokaryotic organisms are well known in the art. Thus, any convenient G-protein mediated signal transduction pathway can be assayed by preparing a chimeric Gα containing the C-terminal residues of a Gα that couples to a PK receptor of the invention, such as Gαq, with the remainder of the protein corresponding to a Gα that couples to the signal transduction pathway it is desired to assay.

An assay to identify compounds that function as PK receptor agonists or antagonists are generally performed under conditions in which contacting the receptor with a known receptor agonist would produce a receptor signal. If desired, the assay can be performed in the presence of a known PKR1 or PKR2 agonist, such as a PK1 or PK2. The agonist concentration can be within 10-fold of the EC₅₀. Thus, an agonist that competes with PK2, PK1 or a PK2/PK1 chimera, for signaling through the PKR2, or indirectly potentiates the signaling activity of PK2, can be readily identified.

Likewise, an antagonist that prevents PK2, PK1 or a PK2/PK1 chimera from binding the PK receptor, or indirectly decreases the signaling activity of the PK receptor also can be identified. Similarly, an antagonist that prevents PK2, PK1 or a PK2/PK1 chimera from binding the PK receptor, or indirectly decreases the signaling activity of PK receptor, also can be identified. The candidate compound can be tested at a range of concentrations to establish the concentration where half-maximal signaling occurs; such a concentration is generally similar to the dissociation constant (Kd) for PK receptor binding.

A binding assay can be performed to identify compounds that are PK receptor agonists or antagonists. In such an assay, a PK receptor polypeptide of the invention can be contacted one or more candidate compounds under conditions in which an agonist such as PK1 or PK2 binds to the PK receptor, and a compound that binds to the PK receptor or a compound that reduces binding of the agonist to the PK receptor can be identified. Contemplated binding assays can involve detectably labeling a candidate compound, or competing an unlabeled candidate compound with a detectably labeled PK agonist, such as a PK2, PK1 or PK2/PK1 chimera. A detectable label can be, for example, a radioisotope, fluorochrome, ferromagnetic substance, or luminescent substance. Exemplary radiolabels useful for labeling compounds include ¹²⁵I, ¹⁴C and ³H. Methods of detectably labeling organic molecules, either by incorporating labeled amino acids into the compound during synthesis, or by derivatizing the compound after synthesis, are known in the art.

In order to determine whether a candidate compound decreases binding of detectably labeled PK, the amount of binding of a given amount of the detectably labeled PK is determined in the absence of the candidate compound. Generally the amount of detectably labeled PK will be less than its K_{d}, for example, 1/10 of its K_{d}. Under the same conditions, the amount of binding of the detectably labeled PK2, PK1 or PK2/PK1 chimera in the presence of the candidate compound is determined. A decrease in binding due to a candidate compound characterized as a PK receptor ligand is evidenced by at least 2-fold less, such as at least 10-fold to at least 100-fold less, such as at least 1000-fold less, binding of detectably labeled PK2, PK1 or PK2/PK1 chimera to PK receptor in the presence of the candidate compound than in the absence of the candidate compound. An exemplary assay for determining binding of detectably labeled PK2, PK1 or PK2/PK1 chimera to PKR2 or PKR1 is the radioligand filter binding assay described in Li et al. Molecular Pharmacology 59:692-698 (2001)).

Either low- and high-throughput assays suitable for detecting selective binding interactions between a receptor and a ligand include, for example, fluorescence correlation spectroscopy (FCS) and scintillation proximity assays (SPA) reviewed in Major, J. Receptor and Signal Transduction Res. 15:595-607 (1995); and in Sterrer et al., J. Receptor and Signal Transduction Res. 17:511-520 (1997)). Binding assays can be performed in any suitable assay format including, for example, cell preparations such as whole cells or membranes that contain a PK receptor of the invention, or substantially purified PK receptor of the invention, either in solution or bound to a solid support.

As used herein, the term “candidate compound” refers to any biological or chemical compound. For example, a candidate compound can be a naturally occurring macromolecule, such as a polypeptide, nucleic acid, carbohydrate, lipid, or any combination thereof. A candidate compound also can be a partially or completely synthetic derivative, analog or mimetic of such a macromolecule, or a small organic molecule prepared by combinatorial chemistry methods. If desired in a particular assay format, a candidate compound can be detectably labeled or attached to a solid support.

Methods for preparing large libraries of compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

The number of different candidate compounds to test in the methods of the invention will depend on the application of the method. For example, one or a small number of candidate compounds can be advantageous in manual screening procedures, or when it is desired to compare efficacy among several predicted ligands, agonists or antagonists. However, it will be appreciated that the larger the number of candidate compounds, the greater the likelihood of identifying a compound having the desired activity in a screening assay. Additionally, large numbers of compounds can be processed in high-throughput automated screening assays.

Assay methods for identifying compounds that selectively bind to or modulate signaling through a PKR2 generally involve comparison to a control. One type of a “control” is a preparation that is treated identically to the test preparation, except the control is not exposed to the candidate compound. Another type of “control” is a preparation that is similar to the test preparation, except that the control preparation does not express the receptor, or has been modified so as not to respond selectively to PK2 or PK1. In this situation, the response of the test preparation to a candidate compound is compared to the response (or lack of response) of the control preparation to the same compound under substantially the same reaction conditions.

A compound identified to be an agonist or antagonist of a PK receptor polypeptide of the invention can be tested for its ability to modulate one or more effects on the function of a cell or animal. For example, a PK receptor agonist or antagonist can be tested for an ability to modulate circadian rhythm function, angiogenesis, gastrointestinal contraction and motility and secretion of gastric acid or pepsinogen, neurological conditions and pain.

Exemplary assays for determining for determining the effect of a compound on circadian rhythm function are described, for example, in Cheng et al. Nature 247:405-410 (2002). Exemplary assays for determining the effect of a compound on angiogenesis are described, for example, in U.S. Pat. No. 5,753,230 and PCT publication WO 97/15666 and U.S. Pat. No. 5,639,725, which describe tumor model systems; Langer et al., Science 193:707-72 (1976); O'Reilly, et al., Cell 79:315-328 (1994); and U.S. Pat. No. 5,753,230. Exemplary assays for determining the effect of a compound on GI contraction and motility are described, for example, in Li et al. Mol Pharmacol. 59(4):692-8 (2001), and Thomas et al., Biochem. Pharmacol. 51:779-788 (1993).

Exemplary assays for determining for determining the effect of a compound on gastric acid or pepsinogen secretion are described, for example, in Soll, Am. J. Physiol 238:G366-G375 (1980); Sol and Walsh, Annu. Rev. Physiol. 41:35-53(1979); Lavezzo et al., Int J Tissue React 6(2):155-165 (1984)) and in isolated gastric mucosae (Rangachari, Am. J. Physiol. 236:E733-E737 (1979), Bunce et al. Br. J. Pharmacol 58:149-156 (1976); and Lavezzo et al., Int J Tissue React 6(2):155-165 (1984)); Howden et al., Aliment Pharmacol Ther 1(4):305-315 (1987); Hirschowitz et al. J. Pharmacol Exp Ther 224(2):341-5 (1983), and Wilson et al. Gig Dis Sci 29(9):797-801 (1984).

Exemplary assays for determining the effect of a compound on neurological conditions include animal models of trauma due to stroke or neural injury are known in the art. One experimental model of stroke involves occluding the right middle cerebral artery and both common carotid arteries of rats for a short period, followed by reperfusion (Moore et al., J. Neurochem. 80:111-118). An experimental model of CNS injury is the fluid percussion injury (FPI) model, in which moderate impact (1.5-2.0 atm) is applied to the parietal cerebral cortex (Akasu et al., Neurosci. Lett. 329:305-308 (2002). Experimental models of spinal cord injury are also used in the art (Scheifer et al., Neurosci. Lett. 323:117-120 (2002). Suitable models for neural damage due to oxidative stress, hypoxia, radiation and toxins are also known in the art.

Exemplary assays for determining the effect of a compound on pain include well-known animal models of pain, such as the Mouse Writhing Assay, the Tail Flick Assay, the Sciatic Nerve Ligation assay, the Formalin Test and the Dorsal Root Ganglia Ligation assay (see, for example, Bennett and Xie, Pain 33:87-107 (1988); and Lee et al., Neurosci. Lett. 186:111-114 (1995); Dewey et al., J. Pharm. Pharmacol. 21:548-550 (1969); Koster et al., Fed. Proc. 18:412 (1959); pain (Malmberg and Yaksh, The Journal of Pharmacology and Experimental Therapeutics 263:136-146 (1992)).

The rhesus monkey prokineticin polypeptides of the invention, as well as a compound identified using a method of the invention, can be formulated and administered in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for a particular therapeutic application in humans can be extrapolated based on the activity of the compound in ex vivo and in vivo assays referenced herein.

The methods of the invention can involve administering a PKR1 or PKR2 agonist or antagonist to prevent or treat a variety of conditions as described herein above. As used herein, the term “administering” when used in reference to a PK receptor agonist or antagonist means providing to or contacting a cell, tissue or animal with the PK receptor antagonist. The term encompasses administering a PK receptor agonist or antagonist in vitro or ex vivo, as to a cell or tissue, which can be a cell or tissue removed from an animal or a cell or tissue placed in or adapted to culture; as well as in vivo, as to an animal. The total amount of a compound can be administered as a single dose or by infusion over a relatively short period of time, or can be administered in multiple doses administered over a more prolonged period of time. Additionally, the compound can be administered in a slow-release matrix, which can be implanted for systemic delivery at or near the site of the target tissue. Contemplated matrices useful for controlled release of compounds, including therapeutic compounds, are well known in the art, and include materials such as DepoFoam™, biopolymers, micropumps, and the like.

A compound can be administered to a mammal by a variety of routes known in the art including, for example, intracerebrally, intraspinally, intravenously, intramuscularly, subcutaneously, intraorbitally, intracapsularly, intraperitoneally, intracisternally, intra-articularly, orally, intravaginally, rectally, topically, intranasally, or transdermally.

Generally, a compound, such as a prokineticin polypeptide and other compounds identified using a method of the invention, are administered to an animal as a pharmaceutical composition comprising the compound and a pharmaceutically acceptable carrier. The choice of pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters. A pharmaceutically acceptable carrier can further contain physiologically acceptable compounds that stabilize the compound, increase its solubility, or increase its absorption. Such physiologically acceptable compounds include carbohydrates such as glucose, sucrose or detrains; antioxidants, such as ascorbic acid or glutathione; chelating agents; and low molecular weight proteins (see for example, “Remington's Pharmaceutical Sciences” 18th ed., Mack Publishing Co. (1990)).

For applications that require the compounds to cross the blood-brain barrier, or to cross cell membranes, formulations that increase the lipophilicity of the compound can be useful. For example, the compounds of the invention can be incorporated into liposomes (Gregoriadis, Liposome Technology, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer. Other approaches for formulating a compound such that it crosses the blood-brain barrier are known in the art and include the use of nanoparticles, which are solid colloidal particles ranging in size from 1 to 1000 nm (Lockman et al., Drug Dev. Ind. Pharm. 28:1-13 (2002)), and peptides and peptidomimetics that serve as transport vectors (Pardridge, Nat. Rev. Drug Discov. 1:131-139 (2002).

The invention provides an antibody selective for squirrel monkey PKR2 polypeptide SEQ ID NO:2 that does not substantially bind to another primate or non-primate PKR2. In addition, the invention provides an antibody selective for chimpanzee PKR2 polypeptide SEQ ID NO:4 that does not substantially bind to another primate or non-primate PKR2. Further provided is antibody selective for rhesus monkey PKR1 polypeptide SEQ ID NO:30 that does not substantially bind to another primate or non-primate PKR1. Also provided is an antibody selective for rhesus monkey PK2 SEQ ID NO:6 that does not substantially bind to another primate or non-primate PK2. Additionally provided is an antibody selective for rhesus monkey PK1 SEQ ID NO:28 that does not substantially bind to another primate or non-primate PK1. The antibodies of the invention can be used, for example, to detect expression of a squirrel or chimpanzee monkey PKR2; rhesus monkey PKR1; rhesus monkey PK1 or rhesus monkey PK2 in research and diagnostic applications. The term “antibody,” as used herein, is intended to include molecules having selective binding activity for an amino acid sequence corresponding to a reference polypeptide of at least about 1×10⁵ M⁻¹, preferably at least 1×10⁷ M⁻¹, more preferably at least 1×10⁹ M⁻¹. The term “antibody” includes both polyclonal and monoclonal antibodies, as well as antigen binding fragments of such antibodies (e.g. Fab, F(ab′)₂, Fd and Fv fragments and the like). In addition, the term “antibody” is intended to encompass non-naturally occurring antibodies, including, for example, single chain antibodies, chimeric antibodies, bifunctional antibodies, CDR-grafted antibodies and humanized antibodies, as well as antigen-binding fragments thereof.

Methods of preparing and isolating antibodies, including polyclonal and monoclonal antibodies, using peptide and polypeptide immunogens, are well known in the art and are described, for example, in Harlow and Lane, Antibodies: A Laboratory Manual, Cold Spring Harbor Laboratory Press (1988). Non-naturally occurring antibodies can be constructed using solid phase peptide synthesis, can be produced recombinantly or can be obtained, for example, by screening combinatorial libraries consisting of variable heavy chains and variable light chains. Such methods are described, for example, in Huse et al. Science 246:1275-1281 (1989); Winter and Harris, Immunol. Today 14:243-246 (1993); Ward et al., Nature 341:544-546 (1989); Hilyard et al., Protein Engineering: A practical approach (IRL Press 1992); and Borrabeck, Antibody Engineering, 2d ed. (Oxford University Press 1995).

### EXAMPLE I

### Cloning of Rhesus Monkey PK2

This example describes cloning of a rhesus monkey prokineticin 2 cDNA.

PCR methods were used to obtain a full-length rhesus monkey PK2 cDNA from reverse-transcribed RNA isolated from rhesus monkey testis. The rhesus monkey PK2 cDNA was amplified with Pfu polymerase using the following primers for PCR: GGCGCCATGAGGAGCCTGTGCTGC (SEQ ID NO:17) and ATTATTCTGATACAGAATTTT (SEQ ID NO:18) followed by nested PCR using the following primers: GGCGCCATGAGGAGCCTGTGCTGC (SEQ ID NO:19) and CTCTTCAAGTGACATTTTCTA (SEQ ID NO:20). The Pfu-catalyzed PCR amplification was carried out for 30 cycles with each cycle consisting of 94° C. denaturing for 30 seconds, 55° annealing for 45 seconds, 68° C. elongation for 90 seconds. The PCR product was cloned into PCRII vector (INVITROGEN CORPORATION, Carlsbad, Calif.) and confirmed by DNA sequencing.

### EXAMPLE II

### Cloning of Squirrel Monkey PKR2

This example describes cloning of a squirrel monkey prokineticin receptor 2 cDNA.

PCR methods were used to obtain a full-length squirrel monkey PKR2 cDNA from reverse-transcribed RNA isolated from squirrel monkey brain. The squirrel monkey PKR2 cDNA was amplified with Pfu polymerase using the following oligonucleotide primers:

ATCACCATGGCAGCCCAGAATGGAAACACCAG (SEQ ID NO:21) and TCACTTCAGCCTGATACAGTCCAC (SEQ ID NO:22). The Pfu-catalyzed PCR amplification was carried out for 50 cycles with each cycle consisting of 94° C. denaturing for 30 seconds, 58° C. annealing for 45 seconds, 68° C. elongation for 90 seconds. The PCR product was cloned into PCRII vector (INVITROGEN CORPORATION, Carlsbad, Calif.) and confirmed by DNA sequencing. For calcium mobilization assays described herein below, the squirrel monkey PKR2 was cloned into pcDNA3.1Zeo (INVITROGEN CORPORATION, Carlsbad, Calif.).

### EXAMPLE III

### Cloning of Chimpanzee PKR2

This example describes cloning of a chimpanzee prokineticin receptor 2 cDNA.

PCR methods were used to obtain a full-length chimpanzee (Pan troglodytes) PKR2 cDNA from reverse-transcribed RNA isolated from chimpanzee salivary gland tissues. The chimpanzee PKR2 cDNA was amplified with Pfu polymerase using the following primers

ATCACCATGGCAGCCCAGAATGGAAACACCAG (SEQ ID NO:23), ATYGCCATTGACAGATATCTYGCCATYGTTCACCCC (Y: T or C)(SEQ ID NO:24), AGATATCTGTCAATGGCRATGGCCAGCAAGGCATTG (R: A or G)(SEQ ID NO:25), and TCACTTCAGCCTGATACAGTCCAC (SEQ ID NO:26). The Pfu-catalyzed PCR amplification was carried out for 45 cycles with each cycle consisting of 94° C. denaturing for 30 seconds, 58° C. annealing for 45 seconds, 68° C. elongation for 90 seconds. The PCR products of primer 1 and 2 (product 1) and those of primer 3 and 4 (product 2) were cloned into PCRII vector (INVITROGEN CORPORATION, Carlsbad, Calif.) and confirmed by DNA sequencing. The complete chimpanzee PKR2 sequence was generated by ligation of product 1 and product 2 with a conserved Eco RV site.

### EXAMPLE IV

### Squirrel Monkey PKR2 Activity is Comparable to Human PKR2 Activity

This example describes that squirrel monkey PKR2 is activated in response to PK1 and has activity comparable to that of human PKR2.

Activity of squirrel monkey PKR2 was assessed by measuring calcium mobilization in response to PK1. For the calcium mobilization assays, squirrel monkey PKR2 cDNA was transiently transfected into CHO cells stably expressing photoprotein aequorin using lipofectamine (INVITROGEN CORPORATION, Carlsbad, Calif.). After 48 hours, the transfected cells were charged in Opti-MEM (INVITROGEN CORPORATION, Carlsbad, Calif.) containing 8 μM of coelenterazine cp at 37° C. for 2 hours. Cells were then detached by brief typsinization and maintained in Hank's Balanced Salt Solution (HBSS) plus 10 mM HEPES (pH 7.5) and 0.1% BSA at about 5×10⁵ cells/ml. 100 μl of cells were injected into the tubes with 20 μl recombinant human PK1 diluted in HBSS plus 10 mM HEPES (pH 7.5) and 0.1% BSA. Luminescence measurements were made using a MONOLIGHT 2010 luminometer (Analytical Luminescence Laboratory, San Diego, Calif.).

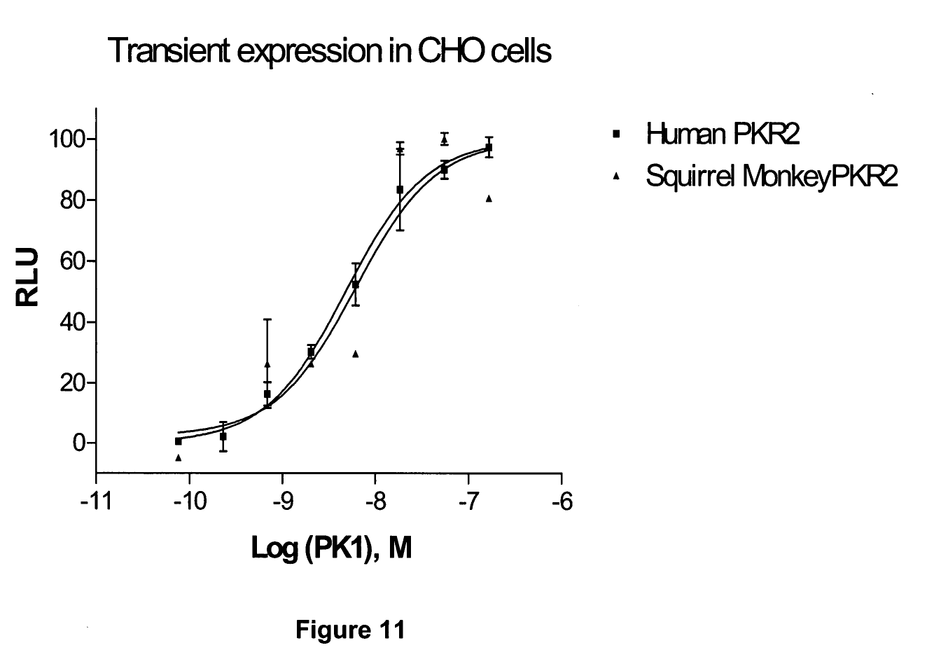

Results of these experiments are shown in FIG. 11, which reveals that squirrel monkey PKR2 has activity comparable to human PKR2 with an EC50 on the order of 10 nM.

### EXAMPLE V

### Cloning of Rhesus Monkey PK1

This example describes cloning of a rhesus monkey prokineticin 1 cDNA.

PCR methods were used to obtain a full-length rhesus monkey PK1 cDNA from reverse-transcribed RNA isolated from rhesus monkey testis. The rhesus monkey PK1 cDNA was amplified with Pfu polymerase using the following primers for PCR: GAGAGGCATCTAAGCAGGCAGTGT (SEQ ID NO:33) and CAATGCACCCAAGAGCCTGTGCCCA (SEQ ID NO:34). The Pfu-catalyzed PCR amplification was carried out for 30 cycles with each cycle consisting of 94° C. denaturing for 30 seconds, 55° annealing for 45 seconds, 68° C. elongation for 90 seconds. The PCR product was cloned into PCRII vector (INVITROGEN Corporation, Carlsbad, Calif.) and confirmed by DNA sequencing.

### EXAMPLE VI

### Cloning of Rhesus Monkey PKR1

This example describes cloning of a rhesus monkey prokineticin receptor 1 cDNA.

PCR methods were used to obtain a full-length rhesus monkey PKR1 cDNA from reverse-transcribed RNA isolated from rhesus monkey testis. The rhesus monkey PKRl cDNA was amplified with Pfu polymerase using the following oligonucleotide primers: CAGATGGAGACCACCATGGGGTTCATG (SEQ ID NO:35), and TTATTTTAGTCTGATGCAGTCCACCTCTTC (SEQ ID NO:36). The Pfu-catalyzed PCR amplification was carried out for 50 cycles with each cycle consisting of 94° C. denaturing for 30 seconds, 58° C. annealing for 45 seconds, 68° C. elongation for 90 seconds. The PCR product was cloned into PCRII vector (INVITROGEN CORPORATION, Carlsbad, Calif.) and confirmed by DNA sequencing. For calcium mobilization assays described herein below, the rhesus monkey PKR1 was cloned into pcDNA3.1Zeo (INVITROGEN Corporation, Carlsbad, Calif.).

### EXAMPLE VII

### Rhesus Monkey PKR1 Activity is Comparable to Human PKR1 Activity

This example describes that rhesus monkey PKR1 is activated in response to PK2 and has activity comparable to that of human PKR1.

Activity of rhesus monkey PKR1 was assessed by measuring calcium mobilization in response to PK2. For the calcium mobilization assays, rhesus monkey PKR1 cDNA was transiently transfected into CHO cells stably expressing photoprotein aequorin using lipofectamine (INVITROGEN Corporation, Carlsbad, Calif.). After 48 hours, the transfected cells were charged in Opti-MEM (INVITROGEN Corporation, Carlsbad, Calif.) containing 8 μM of coelenterazine cp at 37° C. for 2 hours. Cells were then detached by brief typsinization and maintained in Hank's Balanced Salt Solution (HBSS) plus 10 mM HEPES (pH 7.5) and 0.1% BSA at about 5×10⁵ cells/ml. 100 μl of cells were injected into the tubes with 20 μl recombinant human PK2 diluted in HBSS plus 10 mM HEPES (pH 7.5) and 0.1% BSA. Luminescence measurements were made using a MONOLIGHT 2010 luminometer (Analytical Luminescence Laboratory, San Diego, Calif.).

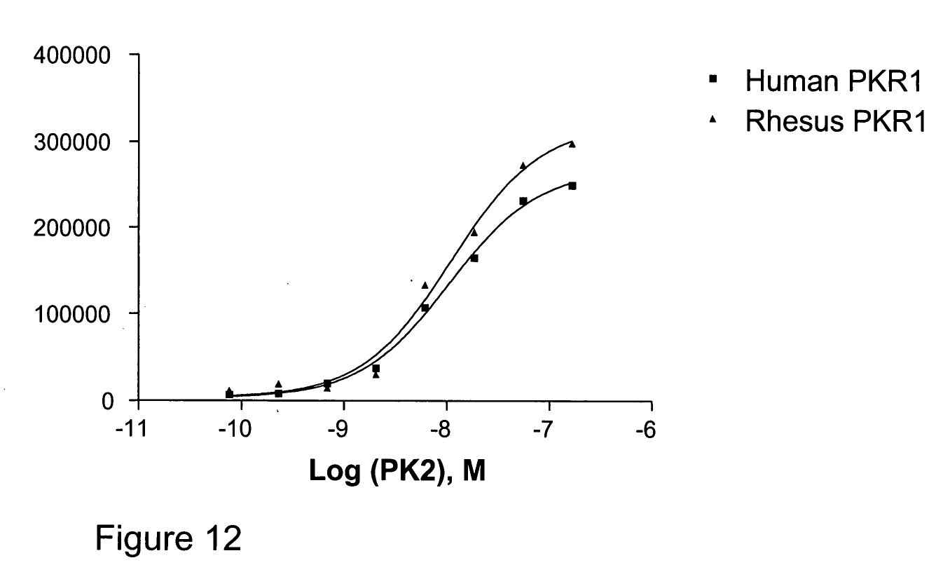

Results of these experiments are shown in FIG. 12, which reveals that rhesus monkey PKRl has activity comparable to human PKR1 with an EC50 on the order of 10 nM.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention.

1. An isolated squirrel monkey prokineticin receptor 2 (PKR2) polypeptide, comprising the amino acid sequence referenced as SEQ ID NO:2.

2-17. (canceled)

18. An isolated chimpanzee prokineticin receptor 2 (PKR2) polypeptide, comprising the amino acid sequence referenced as SEQ ID NO:4.

19-25. (canceled)

26. A method of identifying a PKR2 antagonist comprising:
(a) contacting a PKR2 polypeptide comprising the amino acid sequence referenced as SEQ ID NO:4, with one or more candidate compounds in the presence of a prokineticin, and
(b) identifying a compound that selectively inhibits production of a PKR2 signal, said compound being characterized as an antagonist of said PKR2.

27. The method of claim 26, further comprising determining the ability of said compound to modulate gastrointestinal motility.

28. The method of claim 26, further comprising determining the ability of said compound to modulate pain.

29. The method of claim 26, further comprising determining the ability of said compound to modulate neurogenesis.

30. The method of claim 26, further comprising determining the ability of said compound to modulate circadian rhythm.

31. The method of claim 26, further comprising determining the ability of said compound to modulate angiogenesis.

32. The method of claim 26, wherein said PKR2 signal is calcium mobilization.

33. An isolated nucleic acid molecule encoding a squirrel monkey PKR2 polypeptide, comprising the nucleotide sequence referenced as SEQ ID NO:1.

34-35. (canceled)

36. An isolated nucleic acid molecule encoding a chimpanzee PKR2 polypeptide, comprising the nucleotide sequence referenced as SEQ ID NO:3.

37-38. (canceled)

39. An isolated rhesus monkey PK2 polypeptide, comprising the amino acid sequence referenced as SEQ ID NO:6.

40-45. (canceled)

46. An isolated rhesus monkey prokineticin receptor 1 (PKR1) polypeptide, comprising the amino acid sequence referenced as SEQ ID NO:30.

47-62. (canceled)

63. An isolated nucleic acid molecule encoding a rhesus monkey PKR1 polypeptide, comprising the nucleotide sequence referenced as SEQ ID NO:29.

64-65. (canceled)

66. An isolated nucleic acid molecule encoding a rhesus monkey PK1 polypeptide, comprising the nucleotide sequence referenced as SEQ ID NO:27.

67-68. (canceled)

69. An isolated rhesus monkey prokineticin 1 (PK1) polypeptide, comprising the amino acid sequence referenced as SEQ ID NO:28.

70-71. (canceled)


SEQUENCE LISTING (38 sequences)

SEQ ID NO: 1 - DNA, 1155 bp, Saimiri sciureus
  CDS (1)...(1155)
atggcagcccagaatggaaacaccagttttgcacccaactttaatccaccccaagaccatgcctcctccctctccttcaacttcagttatggtgattacgacctccctatggatgaggatgaggacatgaccaagacccggaccttctttgcagccaagattgtcatcggcattgcactggcaggcatcatgctggtctgtggtgtcggtaactttgtctttatcgctgccctcacccgctataagaagctgcgcaacctcaccaatctgctcattgccaacctggccatctccgacttcctggtggccatcatctgctgcccctttgagatggactactatgtggtccggcagctctcctgggagcatggccacgtgctctgtgcctctgtcaactacctgcgcaccgtctccctctacgtctccaccaatgccttgctggccatcgccattgacagatatctcgccattgttcaccccttgaaaccaaggatgaattatcaaacggcctccttcctgatcgccttggtctggatggtatccattctcattgccatcccatcagcctactttgcaacagaaaccgtcctctttattgtcaagagccaggagaagatcttctgtggccagatctggcccgtggatcagcagctctactacaagtcctacttcctcttcatctttggtgtggagttcgtgggtcctgtggtcaccatgaccctgtgctacgccaggatttcccaggagctctggttcaaggcagtccctgggttccagacagagcagatccgtaagcggctgcgctgccgcaggaagacagtcctggtgctcatgtgcatcctcatggcctacgtgctatgctgggcacccttctatggtttcaccatcgtacgcgacttcttccccaccgtgttcgtaaaggaaaagcactacctcactgccttctacgtggtcgagtgcatcgccatgagcaacagcatgatcaacaccgtgtgcttcgtgacggtcaagaacaacaccatgaagtatttcaagaagatgatgctgctgcactggcgtccctcccagcgggggagcaagtccagtgccgaccttgaccttaagacgaacggggtgcctgccacggaagaggtggactgtatcaggctgaagtga

SEQ ID NO: 2 - protein, 384 aa, Saimiri sciareus
MAAQNGNTSFAPNFNPPQDHASSLSFNFSYGDYDLPMDEDEDMTKTRTFFAAKIVIGIALAGIMLVCGVGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISQELWFKAVPGFQTEQIRKRLRCRRKTVLVLMCILMAYVLCWAPFYGFTIVRDFFPTVFVKEKHYLTAFYVVECIAMSNSMINTVCFVTVKNNTMKYFKKMMLLHWRPSQRGSKSSADLDLKTNGVPATEEVDCIRLK

SEQ ID NO: 3 - DNA, 1155 bp, Pan troglodyte
  CDS (1)...(1154)
atggcagcccagaatggaaacaccagtttcgcacccaactttaatccaccgcaagaccatgcctcctccctctcctttaacttcagttatggtgattatgacctccctatggatgaggatgaggacatgaccaagacccggaccttcctcgcagccaagatcgtcgttggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccatcgccattgacagatatctcgccattgttcaccctttgaaaccacggatgaattatcaaacggcctccttcctgatcgccttggtctggatggtgtccattctcattgccatcccatcggcctactttgcaacagaaaccgtcctctttattgtcaagagccaggagaagatcttctgtggccagatctggcccgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgccgaccttgacctcagaaccaacggggtgcccgccacagaagaggtggactgtatcaggctgaagtga

SEQ ID NO: 4 - protein, 384 aa, Pan troglodyte
MAAQNGNTSFAPNFNPPQDHASSLSFNFSYGDYDLPMDEDEDMTKTRTFLAAKIVVGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPATEEVDCIRLK

SEQ ID NO: 5 - DNA, 1204 bp, Macaca mulatta
  CDS (5)...(329)
cgccatgaggagcctgtgctgcgccccactcctgctcctcctgctgctgccgccgctgctgctcacgccccgcgtcggggacgccgccgtgatcaccggggcttgtgacaaggactcccaatgtggtggaggcatgtgctgtgctgtcagtatctgggttaagagcataaggatttgcacacctatgggcaaactgggagacagctgccatccactgactcgtaaagttccatttgttgggcggaggatgcatcacacttgcccatgtctgccaggcttggcctgtttacggacttcatttaaccgatttatttgtttagcccgaaagtaatcgctttaaagtagaaaccaaatgtgaatagccacatcttatctgtaaagtcttacttgtgattgtgccaaacaaaaaatgtgccagaaagaaatgcttttgcttcctcaactttccaagtaacttttttatctttgagttttaaatgattttttttttaatcgggaattttacttttggatagaaatataaagtgtaaggcattgtggaactggttctcatttccctgtttgtgttttggtttggtttggcttttttcttaaatgtcaaaaacatacccattttcacaaaaatgaggaaaataggaatttgatattttgttagagaaactttttttttcctcaccatcccaagccccatttgtgccccgccacaccataccatacatacatacatacatacatacatacatacatacaacttttggtcccttgcctcttccacctcaaagaatttcaaggcccttaccttactttatttttctccatttctcttccctgctcttgcattttaaagtggtaggtttatctctttgagtttgatggcagaatcgctgatgggaatccagctttttgccggctatttaaatagtgaaaagagtttatatgtgaacttgacactccaaactcctctcatggcgtggacgctgggagtgctgccggacccttcctaaacctgtcactcaagaggacttcggctctgctgttgggctggtgtgtggacagaaggaatggaaagctaaattaatttagtccagatttctaggtttgggtttttctaaaaatgaaagattacgtttacttctttttctttttataaagtttttttttcttagtctcctacttagagatattctagaaaatgtcacttgaa

SEQ ID NO: 6 - protein, 108 aa, Macaca mulatta
  SIGNAL (1)...(27)
MRSLCCAPLLLLLLLPPLLLTPRVGDAAVITGACDKDSQCGGGMCCAVSIWVKSIRICTPMGKLGDSCHPLTRKVPFVGRRMHHTCPCLPGLACLRTSFNRFICLARK

SEQ ID NO: 7 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 8 - protein, 384 aa, M. fascicularis
MAAQNGNTSFAPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQWGSKSSAELDLRTNGVPATEEVDCIRLK

SEQ ID NO: 9 - protein, 81 aa, Homo sapiens
AVITGACDKDSQCGGGMCCAVSIWVKSIRICTPMGKLGDSCHPLTRKVPFFGRRMHHTCPCLPGLACLRTSFNRFICLAQK

SEQ ID NO: 10 - protein, 80 aa, Mus musculus
AVITGACDKDSQCGGGMCCAVSIWVKSIRICTPMGQVGDSCHPLTRKVPFWGRRMHHTCPCLPGLACLRTSFNRFICLAR

SEQ ID NO: 11 - protein, 86 aa, Homo sapiens
AVITGACERDVQCGAGTCCAISLWLRGLRMCTPLGREGEECHPGSHKVPFFRKRKHHTCPCLPNLLCSRFPDGRYRCSMDLKNINF

SEQ ID NO: 12 - protein, 86 aa, Mus musculus
AVITGACERDIQCGAGTCCAISLWLRGLRLCTPLGREGEECHPGSHKIPFLRKRQHHTCPCSPSLLCSRFPDGRYRCFRDLKNANF

SEQ ID NO: 13 - protein, 75 aa, Bufo americanus (toad)
AVITGACDKDVQCGSGTCCAASAWSRNIRFCIPLSGEDCHPASHKVPYDGKRLSSLCPCKSGLTCSKSGEKFKCS

SEQ ID NO: 14 - protein, 77 aa, Anura  Rafinesque (frog)
AVITGACDKDVQCGSGTCCAASAWSRNIRFCIPLGNSGEDCHPASHKVPYDGKRLSSLCPCKSGLTCSKSGEKFKCS

SEQ ID NO: 15 - protein, 80 aa, Serpentes  Linnaeus
AVITGACERDLQCGKGTCCAVSLWIKSVRVCTPVGTSGEDCHPASHKIPFSGQRMHHTCPCAPNLACVQTSPKKFKCLSK

SEQ ID NO: 16 - protein, 81 aa, Homo sapiens
AVITGACERDVQCGAGTCCAISLWLRGLRMCTPLGREGEECHPGSHKVPFFGRRMHHTCPCLPGLACLRTSFNRFICLAQK

SEQ ID NO: 17 - protein, 86 aa, Homo sapiens
AVITGACDKDSQCGGGMCCAVSIWVKSIRICTPMGKLGDSCHPLTRKVPFFRKRKHHTCPCLPNLLCSRFPDGRYRCSMDLKNINF

SEQ ID NO: 18 - DNA, 21 bp, Artificial Sequence
  primer
attattctgatacagaatttt

SEQ ID NO: 19 - DNA, 24 bp, Artificial Sequence
  primer
ggcgccatgaggagcctgtgctgc

SEQ ID NO: 20 - DNA, 21 bp, Artificial Sequence
  primer
ctcttcaagtgacattttcta

SEQ ID NO: 21 - DNA, 32 bp, Artificial Sequence
  primer
atcaccatggcagcccagaatggaaacaccag

SEQ ID NO: 22 - DNA, 24 bp, Artificial Sequence
  primer
tcacttcagcctgatacagtccac

SEQ ID NO: 23 - DNA, 32 bp, Artificial Sequence; identical to SEQ ID NO: 21
  primer

SEQ ID NO: 24 - DNA, 36 bp, Artificial Sequence
  primer
atygccattgacagatatctygccatygttcacccc

SEQ ID NO: 25 - DNA, 36 bp, Artificial Sequence
  primer
agatatctgtcaatggcratggccagcaaggcattg

SEQ ID NO: 26 - DNA, 24 bp, Artificial Sequence
  primer
tcacttcagcctgatacagtccac

SEQ ID NO: 27 - DNA, 762 bp, Macaca mulatta
  CDS (14)...(328)
ccttcaagtgaccatgagaggtgccacgctagtctcaatcatgttcctcctagtaactgtgtctgactgtgctgtgatcacaggggcctgtgagcgggatgtccagtgtggggcaggcacctgctgtgccatcagcctgtggcttcgagggctgcggatgtgtaccccgctggggcgggaaggcgaggagtgccatcctggcagccacaaggtccccttctttaggaaacgcaagcaccatacctgtccttgctcacccaacctgctgtgctccaggttcccagacggcaggtaccgctgctccatggacttgaagaacatcaatttttaggcgcttgccgggtctcaggatacccaccatccttttccccagcactgcctggatttttatttgtgccatgcaacccagctcctgtgactcttccagtccctacgctgactactttgatctctcttgcctagtacacacatatgcacacagggagacatacctcccatcatgacgtggtccccaggctggcctgacgatgtcccagcttggagctgtggtgtgagagatggccagcctggttcccttccctgcttaggctgccagagaggtggtaaatggcagagaggacattccccctcccctcccttcctgggcctgctctccttcctgggccctgtccctctccccacatgtaccccgcggtctgaattggacattcctggcacaggctcttgggtgcattgtgggcacaggctcttgggtgcattgaa

SEQ ID NO: 28 - protein, 105 aa, Macaca mulatta
  SIGNAL (1)...(19)
MRGATLVSIMFLLVTVSDCAVITGACERDVQCGAGTCCAISLWLRGLRMCTPLGREGEECHPGSHKVPFFRKRKHHTCPCSPNLLCSRFPDGRYRCSMDLKNINF

SEQ ID NO: 29 - DNA, 1182 bp, Macaca mullatta
  CDS (1)...(1182)
atggagaccaccatggggttcatggatgacaatgccaccaacacctccaccagcttcctttctgcgctcaaccctcatggagcccatgccgcttccttcccattcaacttcagctatggtgactatgatatgcctttggatgaagatgaggatgtgaccaattcccggacattctttgctgccaagattgtcattgggatggccctggtgggcatcatgctggtctgtggcattggcaacttcgtctttatcgctgctctggtccgctacaagaaactgcgcaatctcaccaacctgctcatcgccaacctggccatctcggatttcctggtggccattgtctgctgcccctttgagatggactactatgtggtgcgccagctctcctgggagcacggccacatcctgtgcacctctgtcaactacctgcgcactgtctctctctatgtctccaccaatgccctgctggccatcgccattgacaggtatctggctattgtccacccgctgagaccacggatgaagtgccaaacagccactggcctgattgccttggtgtggacggtgtccatcctgatcgccatcccttccgcctacttcaccaccgagacggtcctcgtcattgtcaggagccaggaaaagatcttctgcggccagatctggcctgttgaccagcagctgtactacaagtcctacttcctctttatctttggcattgagttcgtgggccccgtgttcaccatgaccctgtgctatgccaggatctcccgggagctctggttcaaggcggtccctggattccagaccgagcagatccgcaagaggctgcgctgccgcaggaagacggtcctggtgcttatgtgcatcctcaccgcctacgtgctgtgctgggcgcccttctacggcttcaccatcgtgcgcgacttcttccccaccgtgttcgtgaaggagaagcactacctcactgccttctacatcgtcgagtgcatcgccatgagcaacagcatgatcaacaccctgtgcttcgtgacagtcaagaacaacaccgccaagtacttcaaaaagatcatgctgctccactggaaggcttcttacaatggcggtaagtccagtgcagacctggacctcaagacaatcgggatgcctgccactgaagaggtggactgcatcagactaaaataa

SEQ ID NO: 30 - protein, 393 aa, Macaca mullatta
METTMGFMDDNATNTSTSFLSALNPHGAHAASFPFNFSYGDYDMPLDEDEDVTNSRTFFAAKIVIGMALVGIMLVCGIGNFVFIAALVRYKKLRNLTNLLIANLAISDFLVAIVCCPFEMDYYVVRQLSWEHGHILCTSVNYLRTVSLYVSTNALLAIAIDRYLAIVHPLRPRMKCQTATGLIALVWTVSILIAIPSAYFTTETVLVIVRSQEKIFCGQIWPVDQQLYYKSYFLFIFGIEFVGPVFTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNNTAKYFKKIMLLHWKASYNGGKSSADLDLKTIGMPATEEVDCIRLK

SEQ ID NO: 31 - protein, 105 aa, Homo sapiens
MRGATRVSIMLLLVTVSDCAVITGACERDVQCGAGTCCAISLWLRGLRMCTPLQREGEECHPGSHKIPFFRKRKHHTCPCLPNLLCSRFPDGRYRCSMDLKNINF

SEQ ID NO: 32 - protein, 393 aa, Homo sapiens [sequence omitted]

SEQ ID NO: 33 - DNA, 24 bp, Artificial Sequence
  PCR primer
gagaggcatctaagcaggcagtgt

SEQ ID NO: 34 - DNA, 25 bp, Artificial Sequence
  PCR primer
caatgcacccaagagcctgtgccca

SEQ ID NO: 35 - DNA, 27 bp, Artificial Sequence
  oligonucleotide primer
cagatggagaccaccatggggttcatg

SEQ ID NO: 36 - DNA, 30 bp, Artificial Sequence
  oligonucleotide primer
ttattttagtctgatgcagtccacctcttc

SEQ ID NO: 37 - protein, 81 aa, Macca mulatta
AVITGACDKDSQCGGGMCCAVSIWVKSIRICTPMGKLGDSCHPLTRKVPFVGRRMHHTCPCLPGLACLRTSFNRFICLARK

SEQ ID NO: 38 - DNA, 24 bp, Artificial Sequence
  PCR primer
ggcgccatgaggagcctgtgctgc